(19) United States
(12) Patent Application Publication
(10) Pub. No.: US 2005/0158856 A1
(43) Pub. Date: Jul. 21, 2005

(54) Methods for producing functional antigen presenting dendritic cells using biodegradable microparticles for delivery of antigenic materials

(76) Inventors: Richard L. Edelson, Westport, CT (US); Carole L. Berger, Bronx, NY (US); Douglas Hanlon, Branford, CT (US); Hong Shen, New Haven, CT (US)

Correspondence Address: MCCARTER & ENGLISH LLP;CITYPLACE I, 185 ASYLUM STREET, HARTFORD, CT, 06103, US

(21) Appl. No.: 10/884,519

(22) Filed: Jul. 1, 2004

Related U.S. Application Data
(63) Continuation-in-part of application No. 10/388,716, filed on Mar. 13, 2003.
Application No. 10/388,716 is a continuation-in-part of application No. 10/066,021, filed on Jan. 31, 2002, now abandoned.
Application No. 10/066,021 is a continuation-in-part of application No. 09/294,494, filed on Apr. 20, 1999, now abandoned.

(51) Int. Cl. C12N 5/08; A61K 9/16; A61K 9/50

(52) U.S. Cl. 435/372; 424/490

(57) ABSTRACT

Methods are provided for producing functional antigen presenting dendritic cells. The dendritic cells are produced by treating an extracorporeal quantity of a subject's blood to induce differentiation of blood monocytes into dendritic cells. The dendritic cells may be exposed to cellular material encapsulated within a biodegradable polymer material to produce the antigen presenting dendritic cells.

The present application is a continuation-in-part of patent application Ser. No. 10/388,716 filed on Mar, 13, 2003, which is a continuation-in-part of patent application Ser. No. 10/066,021 filed on Jan. 31, 2002, which is a continuation-in-part of patent application Ser. No. 09/294,494 filed on Apr. 20, 1999, now abandoned, the entire contents of each of which are hereby incorporated herein by reference.

### FIELD OF THE INVENTION

The present invention relates to methods for producing functional antigen presenting dendritic cells using biodegradable polymer microparticles to deliver encapsulated cellular materials to dendritic cells. The dendritic cells are produced by treating an extracorporeal quantity of a subject's blood using a process referred to herein as transimmunization to induce blood monocytes to differentiate into dendritic cells. The dendritic cells are then exposed to the biodegradable polymers containing cellular materials. The dendritic cells process the cellular materials encapsulated within the biodegradable polymer microparticles and present the materials at the surface of the dendritic cells to induce a cellular immunologic response. The surfaces of the biodegradable polymer microparticles may be modified to target the dendritic cells for delivery of the encapsulated cellular materials. The functional antigen presenting dendritic cells may be administered to a subject to induce cellular immunologic responses to disease causing agents.

### BACKGROUND

Biodegradable polymers have been used for several years to deliver various therapeutic agents to persons requiring treatment. The therapeutic agents typically are encapsulated within the biodegradable polymers which are formed into particles having sizes of 100 μm or less. The biodegradable polymers are administered to a person, and the encapsulated therapeutic agent is released within the body of the patient as the polymer degrades. Biodegradable polymers, both synthetic and natural, can release encapsulated agents over a period of days or weeks, which can have benefits in administration of drugs or other agents. Some of the polymers used for these applications include synthetic polymers such as polylactide-polyglycolide copolymers, polyacrylates and polycaprolactones, or natural polymers such as albumin, gelatin, alginate, collagen and chitosan. Polylactides (PLA) and poly (D, L-lactide-co-glycolide) (PLGA) have been extensively investigated for delivery of therapeutic agents. Biodegradable polymer formulations in medical materials such as sutures have been approved by the FDA for more than three decades.

Biodegradable polymer particles offer several advantages for use in delivering cellular material to dendritic cells to induce cellular immune response from a subject. The polymer particles are biodegradable and biocompatible, and they have been used successfully in past therapeutic applications to induce mucosal or humoral immune responses. Polymer biodegradation products are typically formed at a relatively slow rate, are biologically compatible, and result in metabolizable moieties. Biodegradable polymer particles can be manufactured at sizes ranging from diameters of several microns (microparticles) to particles having diameters of less than one micron (nanoparticles). In the following discussion, the term “microparticles” is used to refer to both microparticles and nanoparticles. To date, molecules successfully encapsulated and released from biodegradable polymers include various drugs, small peptides, proteins (such as antigens), whole bacteria, viruses and plasmid DNA.

Dendritic cells (DCs) are recognized to be powerful antigen presenting cells for inducing cellular immunologic responses in humans. DCs prime both CD8+ cytotoxic T-cell (CTL) and CD4+ T-helper (Th1) responses. DCs are capable of capturing and processing antigens, and migrating to the regional lymph nodes to present the captured antigens and induce T-cell responses. In humans, DCs are a relatively rare component of peripheral blood (<1%), but large quantities of DCs can be differentiated from CD34+ precursors or blood monocytes utilizing cytokine cocktails. Alternatively, by treating an extracorporeal quantity of blood using a process referred to herein as transimmunization, a large number of immature DCs can be induced to form from blood monocytes without the need for cytokine stimulation. These immature DCs can internalize and process materials from disease effectors, such as antigens, DNA or other cellular materials, to induce cellular immunologic responses to disease effectors.

Soluble macromolecules are inherently less stable in solution and less efficiently phagocytosed by dendritic cells than particulate forms of antigen. Therefore, significant interest in particulate systems for delivery of antigen to phagocytes has been generated. The gradual degradation of biodegradable microparticles in aqueous solutions provides an efficient way to control the exposure of encapsulated materials to the environment, and to release encapsulated materials continuously for periods ranging from several hours to several months. The biocompatibility of these microparticles makes them extremely attractive for clinical use. These microparticles have been used principally in isolation. Microparticle encapsulated antigens have rarely been used as a target material specifically for internalization by cultured dendritic cells, and presently only a few proteins or antigenic peptides of known HLA binding specificities have been delivered by any particulate carrier to DC, and only recently by PLGA. Accordingly, use of microparticles to introduce whole cellular materials to dendritic cells may provide improved means for inducing a response to disease effectors. Therefore, biodegradable polymer microparticles, such as for example PLGA conjugates, are excellent candidates for delivery vehicles in immunotherapeutic treatments utilizing cultured dendritic cells.

The efficacy of biodegradable microparticles in delivering material to DCs to induce an immune response may be enhanced by conjugating one or more materials to the surface of the microparticle which will direct the particle to the DC. The surface of the DC includes certain types of receptors which can recognize and bind to proteins and or other molecules. For example, Toll like receptors (TLRs) on the surface of a cell can recognize pathogen-specific molecular patterns (PAMPs). PAMPs are produced only by pathogens and not by host cells, and PAMPs are invariant between microorganisms of a particular class.

Accordingly, the present invention includes biodegradable particles encapsulating cellular material for delivery to dendritic cells, and methods of producing vaccines comprising dendritic cells loaded with cellular materials to induce cellular immunologic responses to disease effectors.

### SUMMARY OF THE INVENTION

The present invention includes biodegradable polymer microparticles encapsulating cellular materials such as, for example, whole cellular antigenic materials from disease effector agents, such as for example, cancer cells. The surface of the polymer microparticles may be unmodified, positively charged, or conjugated with molecules to more efficiently target the microparticles to dendritic cells. For example, in one embodiment of the invention, one or more PAMP (pathogen-associated molecular patterns) are conjugated to the surface of the polymer microparticle prior to the exposure of the microparticle to the dendritic cells. The PAMP targets TLRs (Toll-like receptors) on the surface of dendritic cells, increasing the probability of interaction between the microparticle and dendritic cells. In another embodiment of the invention, a mannose moiety, or other molecules recognized by specific receptors on the dendritic cells, are conjugated to the microparticle to increase the efficiency of particle internalization.

Methods of using the microparticles to induce cellular immunologic responses to disease causing agents are also included. A large number of immature dendritic cells are created by treating a quantity of a patient's blood by flowing the blood through narrow plastic channels in a process referred to herein as transimmunization. Interaction between blood monocytes and the plastic channels induces the blood monocytes to differentiate and form immature dendritic cells. The dendritic cells are then exposed to the microparticles, which are phagocytosed by the dendritic cells. As the dendritic cells mature, they process the cellular materials encapsulated in the microparticle and present the encapsulated cellular materials at the dendritic cell surface. The dendritic cells present the cellular material to the patient's immune system, inducing a cellular immunologic response to the disease effector agents from which the cellular material originated.

In one embodiment of the invention, the immature dendritic cells and the biodegradable microparticles are combined in vitro, incubated together for a period of time, and the resulting dendritic cells are then administered to the patient. In another embodiment, the treated blood and the biodegradable microparticles may be co-administered to the patient, and the dendritic cells can react with the microparticles in vivo.

### BRIEF DESCRIPTION OF THE DRAWINGS

FIG. 1 is a cross-sectional view of a plastic channel containing a blood monocyte from the subject's blood.

FIG. 2 is a cross-sectional view of a plastic channel containing the subject's blood illustrating a blood monocyte adhered to the wall of the plastic channel.

FIG. 3 is a cross-sectional view of a plastic channel containing the subject's blood illustrating a blood monocyte partially adhered to the wall of the channel.

FIG. 4 is an illustration of dendritic cell produced by differentiation of a blood monocyte by the method of the present invention.

FIG. 5 is an illustration of a biodegradable polymer microparticle in the process of being phagocytized by a dendritic cell.

FIG. 6 is an illustration of a dendritic cell which has been reinfused into the subject's bloodstream presenting a class 1 associated peptide antigen to a T-cell.

FIG. 7 is an illustration of the class 1 associated peptide antigen presented on the surface of the dendritic cell as it is received by a complementary receptor site on the T-cell.

FIG. 8 is an illustration of a clone of the activated T-cell attacking a disease-causing cell displaying the class 1 associated peptide antigen.

FIG. 9 is a side view of a plastic treatment apparatus which may be used to induce monocyte differentiation into functional antigen presenting dendritic cells.

FIG. 10 is a view of cross section A-A of the plastic treatment apparatus of FIG. 9.

FIG. 11 is a bar chart showing the increase in immature dendritic cells as indicated by the cell markers CD36/CD38 and MHC Class II/CD83 in samples of blood treated in a cast acrylic device, in an etched acrylic device (4× surface area), in an etched acrylic device (4× surface area) serum free, and using a LPS/Zymogen coated membrane.

FIG. 12 is a bar chart showing the increase in the cell surface MHC Class II cell markers in samples of blood treated in a cast acrylic device, in an etched acrylic device (4× surface area), in an etched acrylic device (4× surface area) serum free, and using a LPS/Zymogen coated membrane.

### DETAILED DESCRIPTION OF PREFERRED EMBODIMENTS

Dendritic cells are highly effective in presenting antigens to responding T-cells; however, dendritic cells normally constitute less than one percent of blood mononuclear leukocytes. Accordingly, a number of in vitro methods have been developed to expand populations of dendritic cells to augment anti-cancer immunity. By exposing increased numbers of dendritic cells to cellular material, such as for example antigens from tumor or other disease-causing cells, followed by reintroduction of the loaded dendritic cells to the patient, presentation of the cellular material to responding T-cells can be enhanced significantly.

For example, culturing blood mononuclear leukocytes for six to eight days in the presence of granulocyte-monocyte colony stimulating factor (GM-CSF) and interleukin-4 (IL-4) produces large numbers of dendritic cells. These cells can then be externally loaded with tumor-derived peptide antigens for presentation to T-cells. Alternatively, the dendritic cells can be transduced to produce and present these antigens themselves. Expanding populations of dendritic cells transduced to produce and secrete cytokines which recruit and activate other mononuclear leukocytes, including T-cells, has shown some clinical efficacy in generating anti-tumor immune responses.

However, transducing cultivated dendritic cells to produce a particular generic antigen and/or additional cytokines is labor intensive and expensive. More importantly, when used to treat a disease such as cancer, this procedure likely fails to produce and present those multiple tumor antigens that may be most relevant to the individual's own cancer. Several approaches have been proposed to overcome this problem. Hybridization of cultivated autologous dendritic cells with tumor cells would produce tetraploid cells capable of processing and presenting multiple unknown tumor antigens. In a second proposed approach, acid elution of Class I and Class II major histocompatability complexes (MHC) from the surface of malignant cells would liberate a broad spectrum of tumor-derived peptides. These liberated peptides could then be externally loaded onto MHC complexes of autologous cultivated dendritic cells.

Because there are limitations to each of these approaches, an improved method of producing functional antigen presenting dendritic cells and for loading the dendritic cells with cellular material from disease causing agents is desirable. The methods described below improve the efficiency, safety and cost-effectiveness of the production of dendritic cells and the loading of the dendritic cells with antigens and cellular materials for presentation to a subject's immune system.

The present invention is based on the convergence of two disparate phenomena: treating blood monocytes in a manner which induces their differentiation into functional dendritic cells, and exposing the dendritic cells to biodegradable polymer microparticles with encapsulated cellular material from disease effector agents, such as, for example, tumor cells from a subject. By combining the treated blood monocytes with the microparticles containing cellular material from disease effector agents for a period of time sufficient to optimize processing and presentation by the dendritic cells of disease associated cellular materials distinctive to the disease effector agents, prior to returning the dendritic cells to the patient, clinically enhanced immunity to the disease agents is achieved.

As used herein, the term “disease effector agents” refers to agents that are central to the causation of a disease state in a subject. In certain circumstances, these disease effector agents are disease-causing cells which may be circulating in the bloodstream, thereby making them readily accessible to extracorporeal manipulations and treatments. Examples of such disease-causing cells include malignant T-cells, malignant B cells, T-cells and B cells which mediate an autoimmune response, and virally or bacterially infected white blood cells which express on their surface viral or bacterial peptides or proteins. Exemplary disease categories giving rise to disease-causing cells include leukemia, lymphoma, autoimmune disease, graft versus host disease, and tissue rejection. Disease associated antigens which mediate these disease states and which are derived from disease-causing cells include peptides that bind to a MHC Class I site, a MHC Class II site, or to a heat shock protein which is involved in transporting peptides to and from MHC sites (i.e., a chaperone). Disease associated antigens also include viral or bacterial peptides which are expressed on the surface of infected white blood cells, usually in association with an MHC Class I or Class II molecule.

Other disease-causing cells include those isolated from surgically excised specimens from solid tumors, such as lung, colon, brain, kidney or skin cancers. These cells can be manipulated extracorporeally in analogous fashion to blood leukocytes, after they are brought into suspension or propagated in tissue culture. Alternatively, in some instances, it has been shown that the circulating blood of patients with solid tumors can contain malignant cells that have broken off from the tumors and entered the circulation. [Kraeft, et al., Detection and analysis of cancer cells in blood and bone marrow using a rare event imaging system, Clinical Cancer Research, 6:434-42, 2000.] These circulating tumor cells can provide an easily accessible source of cancer cells which may be isolated, encapsulated and presented to the dendritic cells in accordance with the method described and claimed herein.

In addition to disease-causing cells, disease effector agents falling within the scope of the invention further include microbes such as bacteria, fungi and viruses which express disease-associated antigens. It should be understood that viruses can be engineered to be “incomplete”, i.e., produce distinguishing disease-causing antigens without being able to function as an actual infectious agent, and that such “incomplete” viruses fall within the meaning of the term “disease effector agents” as used herein.

In the methods described herein, the disease effector agents are presented to the dendritic cells using biodegradable polymer microparticles as delivery vehicles. The disease effector agents are isolated for encapsulation. Any method of isolating disease cells known to those skilled in the art may be used. For example, disease effector agents such as cancer cells may be isolated by surgical excision of cells from a patient. Blood borne disease effector cells may be isolated from an extracorporeal quantity of a subject's blood and the isolated cells may be treated prior to encapsulation.

The isolated disease effector cells may be treated as desired prior to encapsulation. The disease effector agents may be rendered apoptotic prior to encapsulation of cellular material. Apoptosis may be induced by adding photo-activated drugs to the disease cells and exposing the cells to light. Cell death can also be induced by exposure of cells to ionizing radiation, for example by exposure to gamma irradiation or x-rays utilizing devices routinely available in a hospital setting. Cancer cells may be rendered apoptotic by addition of synthetic peptides with the arginine-glycine-aspartate (RGD) motif cell suspensions of the disease-causing cells isolated from the patient's blood, from excised solid tumors or tissue cultures of the same. RGD has been shown (Nature, Volume 397, pages 534-539, 1999) to induce apoptosis in tumor cells, possibly by triggering pro-capase-3 autoprocessing and activation. Similarly, apoptosis could be induced in cells having Fas receptors, by stimulating with antibodies directed against this receptor, in this way sending signals to the inside of the cell to initiate programmed cell death, in the same way that normally Fas ligand does. In addition, apoptosis can be induced by subjecting disease-causing cells to heat or cold shock, certain viral infections (i.e., influenza virus), or bacterial toxins. Alternatively, certain infectious agents such as influenza virus can cause apoptosis and could be used to accomplish this purpose in cell suspensions of disease-causing cells.

Any appropriate biodegradable polymer known to those skilled in the art may be used to encapsulate the disease effector agents. These include, but are not limited to, poly (D, L-lactide-coglycolide, (PLGA), polylactide (PLA), polylactide-polyglycolide copolymers, polyacrylates, polycaprolactone, or polyanhydrides. In a preferred embodiment, PLGA is used as the biodegradable polymer for encapsulating the disease effector agents. The disease effector agents can be encapsulated into the PLGA using double emulsion solvent evaporation techniques known to those skilled in the art. The invention is not limited in this regard, however, and any appropriate means of preparing the polymer microparticles may be used.

The external surface of the biodegradable polymer microparticle may be modified to enhance the ability of the dendritic cell to interact with the microparticle. For example, the outer surface of a polymer microparticle having a carboxy terminus may be linked to PAMPs that have a free amine terminus. Alternatively, if the PAMP has no available reactive termini, the pathogen-associated molecules may be co-encapsulated with the antigenic material. In this way, the PAMP will be exposed to the DC during biodegradation of the particle during the co-incubation period or following engulfment by the DC. The PAMP targets the TLR on the surface of the dendritic cell or signals internally, thereby potentially increasing DC antigen uptake, maturation and T-cell stimulatory capacity. PAMPs conjugated to the particle surface or co-encapsulated may include: unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

In another embodiment, the outer surface of the microparticle may be treated using a mannose amine, thereby mannosylating the outer surface of the microparticle. This treatment may cause the microparticle to bind to the dendritic cell at a mannose receptor on the dendritic cell surface. Alternatively, surface conjugation with an immunoglobulin molecule containing an Fc portion (targeting Fc receptor), heat shock protein moiety (HSP receptor) or phosphatidylserine (scavenger receptors) are additional receptor targets on DC.

The loaded microparticles are exposed to immature dendritic cells, which internalize the microparticles and process the material within the microparticles. In addition, the microparticles may be administered to the patient and the interaction between the microparticles and the dendritic cells may occur in vivo. In a preferred embodiment of the invention, the microparticles are placed in an incubation bag with the immature dendritic cells, and the microparticles are phagocytosed by the dendritic cells during the incubation period. The resulting dendritic cells are then administered to the patient to induce an immune response to the disease causing agent.

Induction of Monocyte Differentiation into Dendritic Cells

As noted above, monocyte differentiation is initiated by exposing the monocytes contained in an extracorporeal quantity of a subject's blood to the physical forces resulting from the sequential adhesion and release of the monocytes on plastic surfaces, such as the surfaces of the channels of a conventional photopheresis device. In one embodiment of the invention, a white blood cell concentrate is prepared in accordance with standard leukapheresis practice using a leukapheresis/photopheresis apparatus of the type well known to those skilled in the art. The white blood cell concentrate includes monocytes, lymphocytes and some red blood cells and platelets. Typically, up to two billion white blood cells are collected during leukapheresis. Assuming that monocytes comprise from about 2% to about 50% of the total white blood cell population collected, approximately 40 million to 1 billion monocytes are present in the white blood cell concentrate.

Following separation by leukapheresis, monocyte differentiation is induced by pumping the blood cell concentrate through a device which has a plurality of plastic channels. Preferably, the plastic channels have a diameter of between about 0.5 mm and 5.0 mm. In one embodiment, a conventional photopheresis apparatus having a channel diameter of 1 mm or less is used. The narrow channel configuration of the photopheresis apparatus maximizes the surface area of plastic to which the blood cell concentrate is exposed as it flows through the photopheresis apparatus. The invention is not limited in this regard, however, and any appropriate device having plastic channels may be used to induce monocyte differentiation.

In one embodiment of the present invention wherein the blood cell concentrate is treated using a conventional photopheresis apparatus, monocyte differentiation is induced by the physical forces experienced by the monocytes as they flow through the plastic channels in the photopheresis apparatus. While the invention is not limited to any particular mechanism, the inventors believe that monocytes in the blood cell concentrate are attracted to the plastic channel walls of the photopheresis apparatus, and the monocytes adhere to the channel walls. The fluid flow through the channel imposes shearing forces on the adhered monocytes that cause the monocytes to be released from the plastic channel walls. Accordingly, as the monocytes pass through the photopheresis apparatus, they may undergo several episodes of adherence to and release from the plastic channel walls. These physical forces send activation signals though the monocyte cell membrane, which results in induction of differentiation of monocytes into immature dendritic cells that are aggressively phagocytic.

Inducing monocytes to form dendritic cells by this method offers several advantages for immunotherapeutic treatment. Because all of the dendritic cells are formed from the monocytes within a very short period of time, the dendritic cells are all of approximately the same age. Dendritic cells will phagocytize apoptotic cells during a distinct period early in their life cycle. In addition, the antigens present in the phagocytized apoptotic cells are processed and presented at the surface of the dendritic cells during a later distinct period. By creating dendritic cells with a relatively narrow age profile, the method of the present invention provides an enhanced number of dendritic cells capable of phagocitizing apoptotic disease effector agents and subsequently presenting antigens from those disease effector agents for use in immunotherapeutic treatment.

Following treatment to initiate differentiation of monocytes, the treated blood cell concentrate may be sequestered for incubation in the presence of biodegradable polymer microparticles containing cellular material to be delivered to the dendritic cells. The incubation period allows the dendritic cells forming and maturing in the blood concentrate to be in relatively close proximity to the biodegradable microparticles, thereby increasing the likelihood that the biodegradable microparticles will be engulfed and processed by the dendritic cells. A standard blood bag may be utilized for incubation of the cells, as is typical in photopheresis. However, it has been found to be particularly advantageous to use a blood bag of the type which does not leach substantial amounts of plasticizer and which is sufficiently porous to permit exchange of gases, particularly CO₂ and O₂. Such bags are available from, for example, the Fenwall division of Baxter Healthcare Corp. under the name Amicus™ Apheresis Kit. Various plasticizer-free blood bags are also disclosed in U.S. Pat. Nos. 5,686,768 and 5,167,657, the disclosures of which are herein incorporated by reference.

The blood cell concentrate and biodegradable microparticles are incubated for a period of time sufficient to maximize the number of functional antigen presenting dendritic cells in the incubated cell population. Typically, the treated blood cell concentrate and biodegradable microparticles are incubated for a period of from about 1 to about 24 hours, with the preferred incubation time extending over a period of from about 12 to about 24 hours. Additional incubation time may be necessary to fully mature the loaded DC prior to reintroduction to the subject. By treating monocytes in the manner described above and then incubating the treated cell population with the biodegradable microparticles, a large number of functional antigen presenting dendritic cells can be obtained. The activated monocytes produce natural cytokines which aid in the differentiation of the monocytes into dendritic cells. Alternatively, a buffered culture medium may be added to the blood bag and one or more cytokines, such as GM-CSF and IL-4, during the incubation period. Maturation cocktails (typically consisting of combinations of ligands such as CD4OL; cytokines such as interferon gamma, TNF alpha, interleukin 1 or prostaglandin E2; or stimulatory bacterial products) may be added to ensure production of fully functional mature DC.

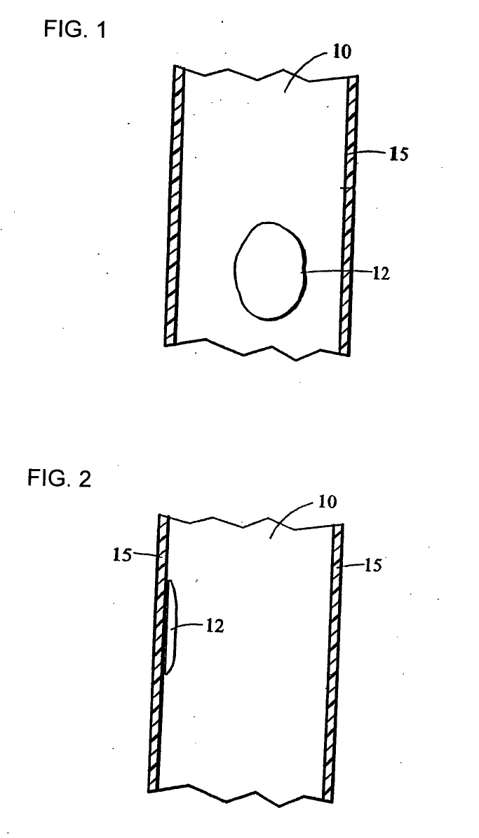

The application of one embodiment of the method described above is illustrated in FIGS. 1 to 7. FIGS. 1 to 7 illustrate treatment of individual cells, but it should be understood that in practice a plurality of blood monocytes will be converted to dendritic cells, and that the plurality of dendritic cells will interact with a plurality of T-cells. Referring to FIG. 1, a plastic channel 10 contains a quantity of the subject's blood, or the blood cell concentrate if the subject's blood is first treated by leukapheresis. The blood contains blood monocytes 12 and is pumped through the plastic channel to induce differentiation of the monocytes into dendritic cells.

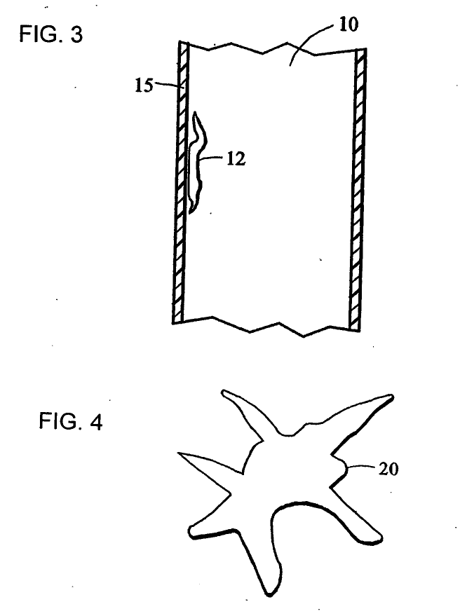

As shown in FIG. 2, as the subject's blood is pumped though the plastic channel, monocytes 12 adhere to the inner walls 15 of the plastic channel 10. Shear forces are imposed on the adhered monocytes by the fluid flowing past the monocytes and, as shown in FIG. 3, the monocytes 12 become dislodged from the wall 15. As the monocytes flow through the plastic channel, they may undergo several episodes of adherence and removal from the channel walls. As a result of the forces experienced by the monocyte, activation signals are transmitted which cause the monocyte to differentiate and form an immature dendritic cell 20, illustrated in FIG. 4. As discussed above, in one embodiment, the plastic channel is part of a conventional photopheresis apparatus.

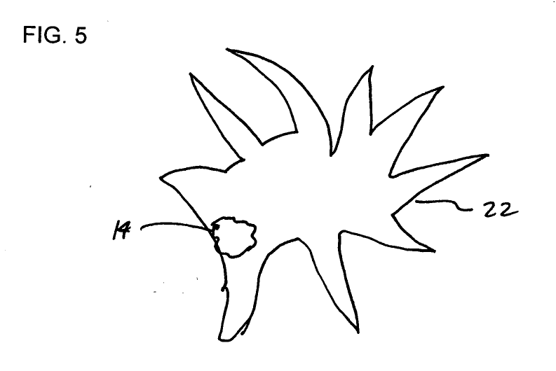

After the blood has been passed through the photopheresis apparatus, the subject's blood is incubated to allow phagocytization of the biodegradable microparticle and subsequent maturation of the dendritic cells. As illustrated in FIG. 5, the dendritic cell 20 ingests the biodegradable microparticle 14 during the incubation period. As the dendritic cell continues to mature during the incubation period, it processes the biodegradable microparticle. At the end of the incubation period, after the dendritic cell digests the biodegradable microparticle, antigens 16 from the cellular materials encapsulated within the biodegradable microparticle are presented at the surface of the dendritic cell 20. After the incubation period, the composition containing the antigen presenting dendritic cells is reinfused into the subject for immunotherapy.

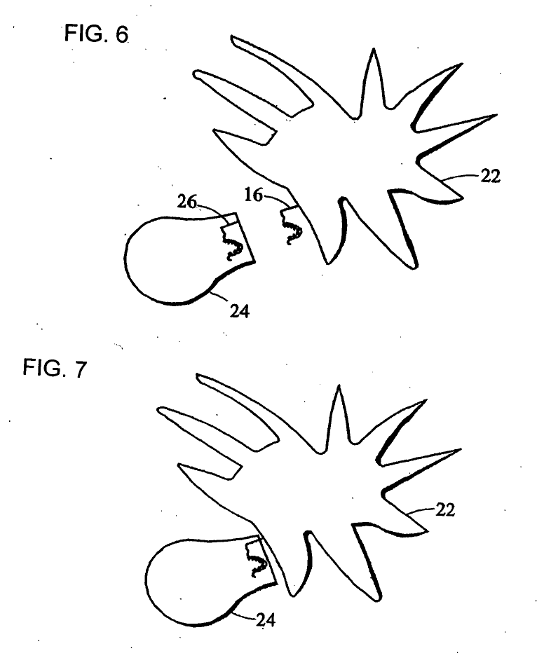

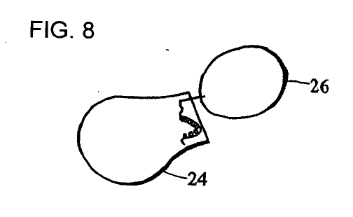

Referring now to FIGS. 6 and 7, which illustrate the dendritic cell after reinfusion into the subject's blood stream, the dendritic cell 22 presents at its surface antigens 16 from the cellular material encapsulated within the biodegradable microparticle to a healthy T-cell 24 which has a receptor site 26 for the antigen 16. When the healthy T-cell 24 receives the antigen from the dendritic cell, as shown in FIG. 7, the healthy T-cell is activated and induces the formation of T-cell clones which will recognize and attack disease effectors displaying the antigen. As a result, as shown in FIG. 8, the healthy T-cell clones 24 of the subject's immune system are triggered to recognize the antigen displayed by the disease effector agent, and to attack and kill disease cells 26 in the subject which display the same antigen.

It should be understood that it is not absolutely necessary to separate the monocytes from the extracorporeal quantity of the patient's blood by leukapheresis prior to treatment. As long as the monocytes contained in the blood are sufficiently exposed to physical forces imposed by flow through plastic channels to initiate differentiation into dendritic cells followed by subsequent incubation, separation of the monocyte population is not required.

Inducing monocyte differentiation according to the method described above provides dendritic cells in numbers which equal or exceed the numbers of dendritic cells that are obtained by expensive and laborious culture of leukocytes in the presence of cytokines such as GM-CSF and IL-4 for seven or more days. The large numbers of functional dendritic cells generated by the method described above provide a ready means of presenting biodegradable microparticles encapsulating selected cellular material, such as for example antigens, plasmids, DNA and are thereby conducive to efficient immunotherapy. Antigen preparations selected to elicit a particular immune response may be derived from, for example, tumors, disease-causing non-malignant cells, or microbes such as bacteria, viruses and fungi. The antigen-loaded dendritic cells can be used as immunogens by reinfusing the cells into the subject or by otherwise administering the cells in accordance with methods known to elicit an immune response, such as subcutaneous, intradermal or intramuscular injection. As described below, it is also possible to generate antigen-loaded dendritic cells by treating and co-incubating monocytes and disease effector agents which are capable of expressing disease associated antigens.

Treatment of Monocytes Using Plastic Treatment Apparatus

In another embodiment of the invention, monocyte differentiation is induced by pumping a blood leukocyte preparation containing monocytes through a plastic treatment apparatus. The plastic treatment apparatus used to treat the monocytes to induce monocyte differentiation may be comprised of any plastic material to which the monocytes will transiently adhere and that is biocompatible with blood leukocyte cells. Examples of materials that may be used include acrylics, polycarbonate, polyetherimide, polysulfone, polyphenylsulfone, styrenes, polyurethane, polyethylene, Teflon or any other appropriate medical grade plastic. In a preferred embodiment of the present invention, the treatment device is comprised of an acrylic plastic.

In the monocyte treatment apparatus, the leukocyte preparation flows through narrow channels. Narrow channels are used to increase the probability and frequency of monocyte contact with the interior plastic surface of the treatment apparatus. The narrow channels also result in flow patterns through the treatment apparatus which impose shearing forces to monocytes transiently contacting or adhering to the interior plastic surfaces of the treatment apparatus.

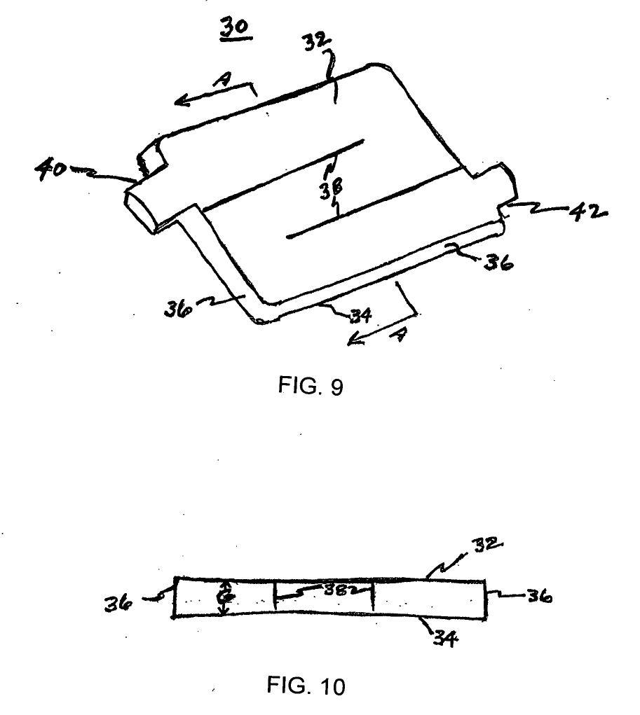

Referring now to FIGS. 9 and 10, one embodiment of a plastic monocyte treatment apparatus is shown. In this embodiment, the treatment apparatus 30 comprises a top plate 32, a bottom plate 34 and side walls 36 to form a box-like structure having a gap, G, between the top plate 32 and the bottom plate 34 to form a narrow channel for flow of blood leukocyte preparations. The top plate 32 and the bottom plate 34 are comprised of a plastic material, such as acrylic or other suitable medical grade plastic as described above.

The side walls 36 of the treatment apparatus may be comprised of the same material as the top plate 32 and the bottom plate 34. Alternatively, the side walls 36 may be comprised of any material, such as for example a rubber, that will form a seal between with the top plate and the bottom plate. The treatment apparatus may have any desired outer shape. For example, the treatment apparatus may have rounded corners, or it may be round or oval.

The top plate 32, bottom plate 34 and side walls 36 may be fastened together using any fastening method known to those skilled in the art. For example, the top plate and bottom plate may be glued to the side walls. Alternatively, bolts, rivets or other fasteners may be used to assemble the top plate, bottom plate and side walls. Gaskets or other sealing materials may be used as necessary to seal the treatment apparatus to prevent leakage.

Internal walls 38 may be provided to direct the flow of the monocytes through the device. The internal walls are typically made of the same material as the top plate and the bottom plate. The internal walls direct the flow of the leukocyte preparation through the treatment apparatus, prevent channeling of flow through the treatment apparatus, and increase the plastic surface area that the monocytes are exposed to within the treatment apparatus. The number of internal walls and the arrangement of the internal walls may be varied to achieve the desired flow pattern through the treatment device. The available surface area may also be increased by including one or more plastic dividers or posts in the flow path through the narrow channels of the plastic treatment apparatus.

The total surface area available for monocyte interaction may also be increased by passing leukocytes through a closed plastic treatment apparatus containing plastic or metal beads. These beads increase the total surface area available for monocyte contact and may be composed of iron, dextran, latex, or plastics such as styrenes or polycarbonates. Beads of this type are utilized commercially in several immunomagnetic cell separation technologies and are typically between 0.001 and 10 micrometers in size. Unmodified beads or those coated with immunoglobulins may also be utilized in this embodiment.

Referring again to FIG. 9, the monocytes enter the treatment apparatus through an inlet connection 40, flow through the treatment apparatus and exit through an outlet connection 42. A pump (not shown) may be used to induce flow through the treatment apparatus, or the treatment apparatus may be positioned to allow gravity flow through the treatment apparatus. The inlet connection 40 and outlet connection 42 may be separate components that are fastened to the treatment apparatus, or they may be made of the same material as the treatment apparatus and formed as an integral part of the top and bottom plates or the side walls.

The top plate 32 and the bottom plate 34 are spaced apart to form a gap G that is preferably between about 0.5 mm and about 5 mm. The total volume of the treatment apparatus is preferably between 10 ml and about 500 ml but may vary depending on the application and blood volume of the mammalian species. Preferably, the leukocyte fraction is pumped through the treatment apparatus at flow rates of between about 10 ml/min and about 200 ml/min. Shearing forces are typically in the range associated with mammalian arterial or venous flow but can range from 0.1 to 50 dynes/cm² . The invention is not limited in this regard, and the volume of the treatment apparatus and the flow rate of the leukocyte preparation through the treatment apparatus may vary provided that sufficient shearing forces are imposed on monocytes contacting the walls of the treatment apparatus to induce monocyte differentiation into functional dendritic cells.

The interior surfaces of the treatment device may be modified to increase the available surface area to which the monocytes are exposed. The increased surface area increases the likelihood that monocytes will adhere to the interior surface of the treatment apparatus. Also, the modified surface may influence the flow patterns in the treatment apparatus and enhance the shearing forces applied to monocytes adhered to the interior surface by the fluid flowing through the treatment apparatus. The interior surfaces of the treatment apparatus may be modified by roughening the surface by mechanical means, such as, for example, by etching or blasting the interior surfaces using silica, plastic or metal beads. Alternatively, grooves or other surface irregularities may be formed on the plastic surfaces during manufacturing. The enclosed exposure area through which the monocytes flow may also consist of a chamber whose contents include beads of various compositions to maximize surface area exposure. The invention is not limited in this regard, and the interior surface or contents of the treatment apparatus may be by any other appropriate method known to those skilled in the art.

In another embodiment of the present invention, plasma and serum proteins are removed from the blood leukocyte preparation prior to passing the leukocytes through the treatment device. Blood proteins, such as hemoglobin, albumins, etc., and cellular components such as platelets or red blood cells, can potentially adhere to the interior plastic surface of the treatment device, thereby creating a surface coating which reduces or prevents monocyte interaction with the plastic surface. By removing serum proteins from the leukocyte preparation prior to pumping the leukocyte preparation through the treatment apparatus, contamination of the plastic surfaces by plasma or serum proteins is reduced or eliminated. Reduction or elimination of this surface contamination increases the available surface area for monocyte interaction.

In this embodiment of the invention, an extracorporeal quantity of blood is treated by leukapheresis to obtain a leukocyte concentrate. The leukocyte concentrate is then further treated to remove plasma and serum proteins from the leukocyte concentrate. The serum may be separated from the leukocytes by performing an additional centrifugal elutriation, density gradient or immunoselection. Centrifugal elutriation may be carried out using a variety of commercially available apheresis devices or one specifically designed for the invention. Density gradients include, but are not limited to, Ficoll Hypaque, percoll, iodoxanol and sodium metrizoate. Immunoselection of purified monocytes may also be utilized to remove contaminating proteins and non-monocyte leukocytes prior to exposure to the device. Alternatively, the leukocyte preparation may be treated by any other method known to those skilled in the art to separate mononuclear cells from other blood components Following removal of serum or plasma components, the leukocyte preparation is pumped through a plastic monocyte treatment apparatus as described above to induce monocyte differentiation into dendritic cells. After the leukocyte preparation is pumped through the treatment apparatus, it is incubated for an appropriate period of time to allow the treated monocytes to differentiate into functional dendritic cells. During this time, immature dendritic cells may be loaded with exogenous antigens including those from whole cells, proteins or peptides. The treated monocytes are typically incubated for a period of between about 12 hours and about 36 hours.

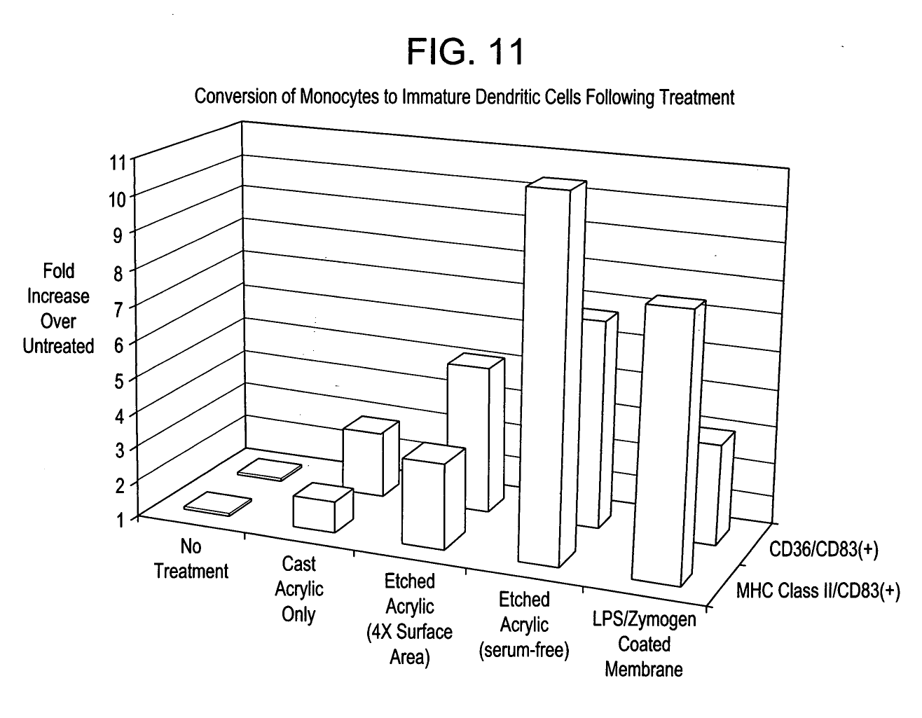

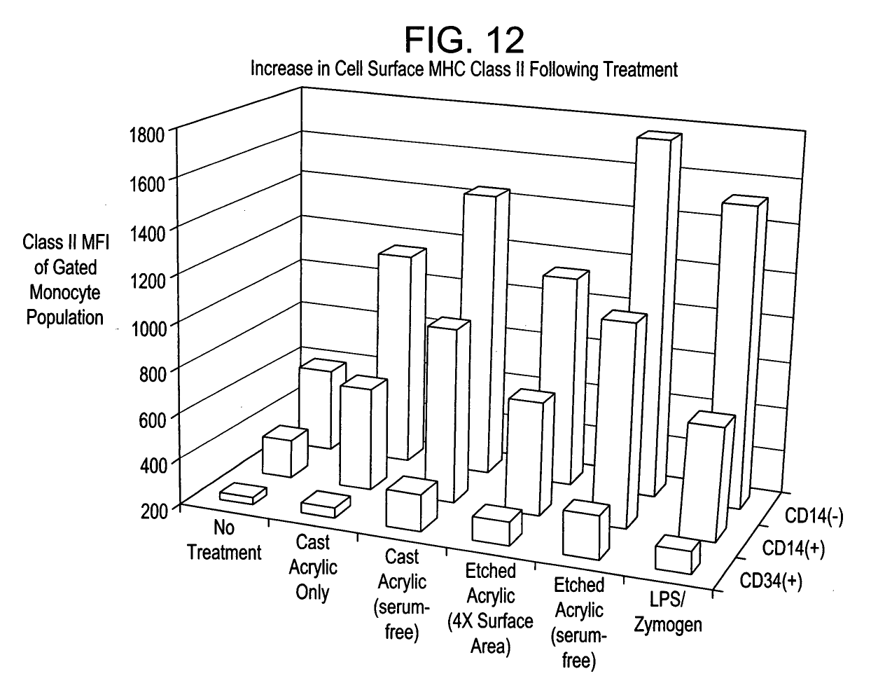

The efficacy of the methods described above are demonstrated by the data shown in FIGS. 11 and 12. This data was obtained using a small plastic treatment apparatus to treat samples of peripheral blood containing monocytes. The treatment apparatus used in these tests had acrylic top plates and bottom plates which were bolted together. The treatment apparatus had a single channel of 30 by 3 cm dimension, 1 mm interplate gap and a total void volume of approximately 10 ml. The leukocyte concentrate was pumped through the treatment apparatus at a flow rate of about 50 ml/minute for 30 minutes. The treated cells incubated overnight to allow differentiation of monocytes into functional dendritic cells.

The data illustrated in FIGS. 11 and 12 was obtained by treating peripheral blood in (1) a treatment apparatus having an unmodified cast acrylic panel; (2) a treatment apparatus having an acrylic panel etched with silica beads to increase the surface area of the panel by a factor of approximately four; and (3) a treatment apparatus having an etched acrylic panel and serum-free peripheral blood monocytes (PBMC) isolated over Ficoll Hyplaque. The conversion of blood monocytes to immature dendritic cells was measured by using previously established markers of dendritic cell development, including cell surface MHC class II and CD36 and intracellular production of CD83.

As shown in FIGS. 11 and 12, treatment of peripheral blood in a cast acrylic treatment apparatus approximately doubled the population of immature dendritic cells in the samples as compared to untreated blood. When the interior surface of the acrylic treatment apparatus was etched to increase the surface area, treatment of the peripheral blood approximately tripled the population of immature dendritic cells as compared to untreated blood. Treatment of peripheral blood with the serum removed prior to treatment increased the population of dendritic cells by a factor of up to eight as compared to untreated blood.

These results demonstrate that treatment of peripheral blood monocytes by pumping the monocytes through a plastic treatment apparatus having narrow channels is an effective method of inducing monocyte differentiation into functional dendritic cells. Etching the surface of the treatment apparatus and removing plasma and serum from the peripheral blood being treated can further enhance the population of dendritic cells obtained.

In another embodiment of the present invention, peripheral blood monocytes are pumped through a treatment apparatus similar to that described above, with at least one interior surface of the treatment apparatus comprising a membrane or surface coated with either pathogen associated inflammatory molecules such as LPS and Zymogen, or with known monocyte ligands that interact with monocyte adhesion molecules (including, for example, E-selectin, ICAM-1, Fract\dot{a}lkine or MCAF/CCC2). As the monocytes in the peripheral blood flow through the treatment apparatus, the monocytes are exposed to these proteins. The stimulatory surface interaction between these molecules and the monocytes induces monocyte differentiation into functional dendritic cells. As shown in FIG. 11, the dendritic cell population in peripheral blood samples treated by exposing the monocytes to an LPS/Zymogen coated membrane is comparable to the increase population observed by treatment of a serum-free blood in an etched acrylic treatment apparatus. It will be recognized that this embodiment of the invention is not limited to use of LPS and Zymogen, the treatment apparatus may include any protein that can be crosslinked to solid supports such as nylon membranes or plastic surfaces and will interact with blood monocytes to induce differentiation into functional dendritic cells. Proteins which can be absorbed to solid supports and used to induce monocyte differentiation include, but are not limited to, inflammatory molecules, adhesion molecules, cytokines, chemokines or serum proteins known to affect leukocyte adhesion and activation.

The dendritic cells formed by the methods described above can be co-incubated with biodegradable polymer microparticles as described previously. The dendritic cells will phagocytize the biodegradable microparticles, process the material contained within the microparticles, and induce an immune response to disease effectors.

In another embodiment of the present invention, in order to bring the induced dendritic cells into physical contact with biodegradable polymer microparticles, an extracorporeal quantity of blood may be treated using a plastic treatment apparatus to induce monocyte differentiation, and the treated monocytes may be reintroduced to the subject without overnight incubation. The biodegradable polymer microparticles containing disease effector agents are administered to the subject, and the interaction between the dendritic cells and the microparticles occurs in vivo.

Among the advantages of this embodiment of the invention are that the treatment time is reduced, as no incubation is required after treatment of the extracorporeal quantity of blood; If desired, the treatment can be combined with radiation or chemotherapeutic treatments in one procedure, thereby reducing the number of times a particular subject must appear for treatment.

After the extracorporeal quantity of the patient's blood has been treated in the plastic treatment device device, the composition is incubated for a period of from about 1 to about 48 hours, most preferably from about 12 to about 24 hours. During this period, the dendritic cells phagocytize microparticles containing the apoptotic disease effector agents and present antigens from the phagocytized cells at their surface, where they will be recognized by T-cells in the patient's immune system, thereby inducing an immunological response to the disease effector agents in the patient.

In another embodiment of the method, immature dendritic cells may be produced utilizing the methods described above, and the immature dendritic cells may be administered to the patient. Biodegradable polymer microparticles containing encapsulated cellular materials may be administered to the patient by intradermal injection or other appropriate method. The immature dendritic cells encounter the biodegradable microparticles in vivo, and process the particles as described above to induce an immunologic response.

Induction of Monocyte Differentiation Using a Packed Column

In another embodiment, monocyte differentiation may be induced by exposing monocytes contained in an extracorporeal quantity of the patient's blood to physical perturbation, in particular to the forces exerted on the monocytes by their sequential adhesion to and release from plastic surfaces as they flow through narrow channels formed by the packing in a column that may be used to treat a patient's blood. In one embodiment of the invention, a white blood cell concentrate is prepared from an extracorporeal quantity of the patient's blood in accordance with standard leukapheresis practice known to those skilled in the art, and as discussed above. The invention is not limited in this regard, and an extracorporeal quantity of a patient's blood may be treated directly without first obtaining a white blood cell concentrate by leukapheresis.

Following separation by leukapheresis, monocyte differentiation is induced by pumping the blood cell concentrate through a packed column containing plastic beads or some other appropriate packing materials that channels flow through the column. The column packing creates a tortuous flow path through the column, resulting in physical forces imposed on the monocytes to induce differentiation of the monocytes into dendritic cells. In this embodiment, the body of the column may be comprised of a plastic material to which the monocytes may adhere, or the body of the column may be substantially comprised of a non-plastic material and have an interior lining or coating of plastic. The packing in the column is a material, typically a plastic material, to which blood monocytes will adhere temporarily as the blood flows through the column.

Materials used for column packing may include dextran, latex, cellulose acetate, acrylics, polycarbonate, polyetherimide, polysulfone, styrenes, polyurethane, polyethylene and Teflon. The packing is preferably in the form of spherical beads, although any shape may be used that will produce flow of the monocytes through channels. The beads are preferably have an average diameter between 0.001 and 10 microns. There are a number of commercially available columns, such as the Adacolumn® sold by Japan Immunoresearch Laboratories Co., Ltd., that may be used to treat blood monocytes to induce differentiation into dendritic cells.

The total volume of the column is preferably between 10 ml and about 500 ml, but it may vary depending on the treatment application. Preferably, the leukocyte fraction is pumped through the treatment apparatus at flow rates of between about 10 ml/min and about 200 ml/min. Preferably, the blood is pumped through the treatment apparatus at a sufficient flow rate to produce shearing forces of monocytes adhered to the column packing in the range typically associated with mammalian arterial or venous flow, although the shearing forces can range from 0.1 to 50 dynes/cm2.

In another embodiment of the method of the present invention, target disease cells, such as for example CTCL cells, are coated with CD3 magnetic beads. In this embodiment, the column is packed with an iron matrix, as in the magnetic bead column made by Miltenyi, and passage through the small spaces in the matrix serve to activate the monocytes. The CD3 antibody binding renders the disease cells apoptotic. The leukocyte concentrate and the coated disease cells are concurrently passed through a packed column as described above through a magnetic field that binds the bead-coated disease cells. Free monocytes in the leukocyte concentrate

The extracorporeal quantity of blood is typically pumped through the treatment device. The extracorporeal quantity of blood may be caused to flow through the plastic channels by gravity, by pressure or by any other technique which will cause the blood to flow through the plastic channels.

While the invention is not limited to any particular mechanism of monocyte differentiation, it is believed that monocytes in the blood cell concentrate are attracted to the plastic surfaces of the channel walls or the column packing, and the monocytes adhere to the plastic surfaces. The fluid flow through the treatment device imposes shearing forces on the adhered monocytes that cause the transiently and incompletely adherent monocytes to be released from the plastic channel walls. Accordingly, as the monocytes pass through the treatment device, they may undergo numerous episodes of transient adherence to and release from the plastic channel walls. These physical forces send activation signals though the monocyte cell membrane, which results in induction of differentiation of monocytes into functional dendritic cells. Preliminary evidence suggests that interaction of monocyte μ-glycoprotein with the plastic surface may contribute to the monocyte entry into the dendritic cell maturational pathway. Therefore, it may be possible to induce monocyte-to-dendritic cell maturation by direct interaction with monocyte P- glycoprotein, without use of a plastic flow system.

After monocyte differentiation to dendritic cells has been induced, the treated blood is incubated for a sufficient period of time to allow the dendritic cells to develop to the desired stage of maturity prior to truncation of maturation. Incubation of the recipient dendritic cells is performed using techniques known to those skilled in the art. In a preferred embodiment, incubation is performed at approximately 37 degrees Centigrade in a standard incubator containing a gaseous environment having approximately 5% carbon dioxide and approximately 95% oxygen, with only trace amounts of other gases.

### EXAMPLE

Biodegradable Nanoparticle Mediated Delivery of Tumor Antigens to Differentiating Dendritic Cells

In one embodiment of the present invention, whole tumor tissue is encapsulated in a biodegradable polymer and combined with immature dendritic cells produced by treating blood monocytes to induce differentiation of the monocytes. Macroscopically non-necrotic tumor tissue is obtained under sterile conditions directly from the operating room or from the surgical pathology section following pathological evaluation. Alternatively, cellular material from blood-borne or lymphoid associated malignancies, such as those associated with leukemias and lymphomas, can be isolated from peripheral blood or lymph tissue and purified. The tumor tissue is weighed and immediately snap frozen in liquid nitrogen. The tumor tissue remains frozen until further processing.

The frozen tumor tissue may be prepared for encapsulation using mechanical means, chemical means, or any other appropriate means known to those skilled in the art. For example, the whole frozen tumor tissue may be mechanically pulverized into a powder having nanometer or micrometer-sized particles, and the frozen powder directly encapsulated into the biodegradable polymer particles as described below. Alternatively, cellular material from the tissue may be brought back to about 4° C. and disrupted using a detergent or other chemical means (for example, detergents such as Triton X-100, NP-40 and digitonin or the denaturant urea) or physical means (for example, sonication or freeze-thaw lysis). After cell lysis, a protein or nucleic acid fraction of the tissue may be isolated in a “lysate”. This lysate may consist of, but is not limited to, soluble or insoluble proteins, proteins associated with certain chaperone molecules such as heat shock proteins or nucleic acids such as DNA or RNA.

In another embodiment of the invention, the tumor tissue is not cryopreserved but instead dissociated into a viable single cell suspension by mechanical dissociation, for example by utilizing a device such as a Becton Dickenson “Medimachine”, or by chemical means by enzymes previously utilized in the dissociation of whole tissues, such as for example trypsin, hyalluronidase, collagenase, or any other appropriate enzyme known to those skilled in the art. These cell suspensions may be utilized immediately as cellular materials for nanoparticle encapsulation or may be further cultured to increase cell number or to select for specific cell types.

To increase the antigenicity of the tumor material, whole tumor tissue or dissociated cells may be further treated by additional means to induce cell death prior to nanoparticle encapsulation. For purposes of the methods presented herein, cell death may be induced by any appropriate method known to those skilled in the art. One type of cell death, classified as apoptosis (programmed cell death or PCD), is induced by signaling through cell-death associated surface receptors or exposure to a variety of chemical, radioactive or light energy sources known to those skilled in the art. A second type of cell death, necrosis, is induced by noxious stimuli such as for example non-physiological temperature fluctuations or culture conditions. Methodologies for inducing cell death and their effects on immunogenicity are described in Melcher and Vile, “Apoptosis or necrosis for tumor therapy.”

An example of a double emulsion encapsulation method is described below. The invention is not limited in this regard, and any appropriate method of encapsulation known to those skilled in the art may be used in the methods described herein.

To encapsulate the treated tumor tissue material in a biodegradable microparticle, an aqueous solution of the treated tumor tissue material is mixed with a biodegradable polymeric material, such as for example with Poly (lactide-co-glycolide) (PLGA) methylene choloride solution, while vortexing to form first emulsion. The first emulsion is sonicated for 10-30 seconds on ice. Following the sonication step, the first emulsion is added to 1% poly(vinyl alcohol) (PVA) solution while vortexing the mixture to form second emulsion. The second emulsion is sonicated for 10-30 seconds on ice. Following sonication, the second emulsion is poured into 0.3% PVA solution and stirred for 3 hours to form the desired nanoparticles. The resulting solution is centrifuged to collect nanoparticles. The nanoparticles are washed with sterile water three times and freeze-dried for 24 hours. The nanoparticles can be stored at <−20° C. in a dry box.

Nanoparticles containing treated tumor tissue material can be utilized immediately in vaccination protocols as discussed below. Alternatively, the nanoparticles can be stored indefinitely at −20° C. or below for later use, for example when additional treatment may be necessary following metasteses or recurrent disease.

In a preferred embodiment of the invention, the nanoparticles are introduced to immature dendritic cells (DC) created from blood monocytes using the “transimmunization” procedure. The immature DC phagocytize the nanoparticles, process the antigenic material contained in the nanoparticles, and present the antigenic materials on the surface of the DC. The antigen-loaded DC are returned to the patient in the form of an anti-tumor vaccine to induce an immune response in the subject to tumor cells having the same antigenic markers. Alternatively, DC created by other means, such as those differentiated from blood monocytes or other progenitors in the presence of cytokines, could take up the particles and represent tumor antigens.

In another embodiment of the invention, particles could be used without cellular adjuvants and instead be directly injected or exposed to dermal, mucosal or lymph tissue where they would be phagocytized by resident cells and presented to the immune system. In addition, particle formulations designed to rapidly dissolve under physiological conditions could release tumor materials into selected tissues where the protein or nucleic acid contents could be taken up by phagocytosis, receptor-mediated endocytosis or pinocytosis by resident immune cells.

It should be understood that the present invention is not limited to treatments involving only encapsulated tumor cells. Other disease-causing cells may be encapsulated in biodegradable polymers for presentation to dendritic. Such cells include, for example, disease associated reactive T-cells and B-cells in autoimmune disorders, virally or bacterially infected cells in diseases such as HIV, hepatitis or papilloma associated cancers, or any cell chronically infected with known pathogens.

1. A method of producing functional antigen presenting dendritic cells comprising the steps of:
(a) obtaining an extracorporeal quantity of a subject's blood;
(b) treating the extracorporeal quantity of blood to obtain a leukocyte concentrate;
(c) treating the leukocyte concentrate by pumping the leukocyte concentrate through a plastic treatment apparatus having at least one channel having a diameter of 1 mm or less to induce differentiation of the monocytes into dendritic cells;
(d) obtaining disease effector cells from the subject;
(e) treating the disease effector cells to obtain cellular materials for encapsulation;
(f) encapsulating the cellular material in a biodegradable polymeric material to form a microparticle;
(g) incubating the dendritic cells and the encapsulated cellular material together for a sufficient period of time to allow the dendritic cells to internalize the biogradable polymer microparticles.

2. The method of claim 1, wherein the disease effector cells are cancer cells removed from a tumor within the subject.

3. The method of claim 1, wherein the disease effector cells are selected from the group consisting of malignant T-cells, malignant B-cells, T-cells which mediate an autoimmune response, and B-cells which mediate an autoimmune response.

4. The method of claim 1, wherein the biodegradable polymeric material is selected from the group consisting of poly (D, L-lactide-coglycolide, (PLGA), polylactide (PLA), polylactide-polyglycolide copolymers, polyacrylates, polycaprolactone, or polyanhydrides.

5. The method of claim 1, wherein the biodegradable polymeric material is PLGA.

6. The method of claim 5, wherein the cellular material is encapsulated in the PLGA by double emulsion solvent evaporation.

7. The method of claim 6, wherein a pathogen-specific molecular pattern having a free amine terminus is linked to a carboxy terminus on the surface of the PLGA microparticle.

8. The method of claim 2, wherein the step of treating the cancer cells to obtain cellular material for encapsulation comprises freezing and pulverizing the cancer cells.

9. The method of claim 1, wherein the dendritic cells and the encapsulated disease effector cells are incubated together between about 1 hour and about 48 hours.

10. The method of claim 1, wherein the leukocyte concentrate is treated to remove substantially all plasma and serum proteins from the leukocyte concentrate.

11. The method of claim 1, wherein the treatment apparatus comprises a plastic selected from the group consisting of acrylics, polycarbonate, polyetherimide, polysulfone, polyphenylsulfone, styrenes, polyurethane, polyethylene and Teflon.

12. The method of claim 11, wherein the surface of the plastic channel exposed to the leukocyte concentrate is mechanically treated to increase to increase the surface area.

13. The method of claim 10, wherein the step of treating the leukocyte concentrate to reduce the quantity of plasma and serum proteins in the leukocyte concentrate comprises treating the leukocyte concentrate using a density gradient.

14. The method of claim 10, wherein the step of treating the leukocyte concentrate to reduce the quantity of plasma and serum proteins in the leukocyte concentrate comprises treating the leukocyte concentrate by immunoselection.

15. The method of claim 1, wherein the treatment apparatus comprises a rectangular top plate fixedly attached to a plurality of side walls; a bottom plate fixedly attached to the plurality of side walls opposite the top plate; an inlet connection fixedly attached to one side wall wherein the inlet connection allows fluids to flow into the treatment apparatus; and an outlet connection fixedly attached to a second side wall wherein the outlet connection allows fluids to flow out of the treatment apparatus.

16. The method of claim 15, wherein the top plate and the bottom plate of the treatment apparatus are spaced apart at a distance of between about 0.5 mm and about 5 mm.

17. The method of claim 15, wherein the treatment apparatus has a volume of between about 10 ml and about 500 ml.

20. The method of claim 15, wherein the leukocyte concentrate is pumped through the treatment apparatus at a flow rate of between about 10 ml/min and about 200 ml/min.

21. The method of claim 15, wherein the leukocyte concentrate is pumped through the treatment apparatus to induce shearing forces of between about 0.1 dyne/cm² to about 50 dynes/cm² on monocytes adhering to the walls of the at least one plastic channel.

22. A method of producing functional antigen presenting dendritic cells comprising the steps of:
(a) obtaining tumor cells from a subject;
(b) freezing the tumor cells in liquid nitrogen;
(c) mechanically pulverizing the frozen tumor cells into a powder;
(d) mixing the cell powder with a biodegradable polymeric material while vortexing to form a first emulsion;
(e) sonicating the first emulsion for 10-30 seconds on ice;
(f) adding the first emulsion to a solution containing about 1% poly(vinyl alcohol) while vortexing to form a second emulsion;
(g) sonicating the second emulsion on ice for 10-30 seconds;
(h) adding the second emulsion to a solution containing about 0.3% poly(vinyl alcohol) and stirring the resulting mixture for about 3 hours;
(i) centrifuging the stirred mixture and collecting the nanoparticles;
(j) washing the nanoparticles with sterile water and freeze drying the nanoparticles for about 24 hours;
(k) supplying immature dendritic cells; and
(l) combining the nanoparticles with the dendritic cells and co-incubating the nanoparticles and the dendritic cells for a sufficient time to allow a substantial portion of the dendritic cells to phagocytize the nanoparticles.
